(19) United States
(12) Patent Application Publication
(10) Pub. No.: US 2025/0299807 A1
(43) Pub. Date: Sep. 25, 2025

(54) SYSTEM AND METHOD FOR MANAGING AND CONTROLLING USER AND FACILITY RESOURCES

(71) Applicant: SMIRTA INNOVATIONS INC., Holmdel, NJ (US)

(72) Inventors: Shridar IYENGAR, Morganville, NJ (US); Paramasiviah HARSHAVARDHANA, Marlboro, NJ (US); Ram IYENGAR, Holmdel, NJ (US); Bhaskar ANEPU, Allentown, PA (US)

(21) Appl. No.: 19/231,969

(22) Filed: Jun. 9, 2025

Related U.S. Application Data
(63) Continuation of application No. 18/057,769, filed on Nov. 22, 2022.
(60) Provisional application No. 63/282,045, filed on Nov. 22, 2021.
(60) Provisional application No. 63/296,886, filed on Jan. 6, 2022.

(51) Int. Cl. G16H 40/20; G16H 10/60

(52) U.S. Cl. CPC G16H 40/20; G16H 10/60

(57) ABSTRACT

A system and method for facilitating patient scheduling at a healthcare facility is disclosed. The method includes receiving a request from one or more electronic devices associated with a scheduler to schedule an appointment of a patient, obtaining treatment information, provider information and resource information from an EHR system, and obtaining one or more available slots of provider from the EHR system. Furthermore, the method includes determining one or more optimal treatment times for the treatment date for the treatment profile of the patient and outputting the determined one or more optimal treatment times for the treatment date along with relevant patient information on user interface screen of the one or more electronic devices associated with the scheduler.

### CROSS-REFERENCE TO RELATED APPLICATIONS

This application is a continuation of and claims priority from U.S. patent application Ser. No. 18/057,769, filed Nov. 22, 2022, which claims the benefit of priority from U.S. Provisional Patent Application No. 63/282,045, filed Nov. 22, 2021, and U.S. Provisional Patent Application No. 63/296,886, filed Jan. 6, 2022, whereby the entirety of each application are incorporated herein by reference.

### FIELD OF DISCLOSURE

Embodiments of the present disclosure relate to patient treatment systems, and more particularly relates to a system and method for facilitating patient scheduling at a healthcare facility.

### BACKGROUND

Patient scheduling is a process of assigning individual patients and/or patients' activities to a specific time and/or healthcare resources. Generally, patient scheduling at healthcare facilities is extremely complex. The volume of patients on any specific day in the future is highly variable. There is also the impact of cancellations, add-ons, and no-shows, and a mix of treatment durations for a given day. This becomes a central issue of treatment scheduling that creates a challenge for schedulers. It creates a logistical challenge that is beyond the capacity of a normal human mind to solve, specifically in a short amount of time with limited information that is available at the time of scheduling a patient. Further, sub-optimal scheduling tends to result in long patient wait times, imbalanced treatment chair utilization across a given day, and uneven nurse load resulting in high stress levels.

For example, cancer treatment scheduling can be incredibly complex due to a multitude of services involved in the process and a wide variation in treatment durations. From a healthcare service provider point of view, nothing can be more stressful than caring for sick patients. Peak hours and days when the volume of patients and number of procedures surpass staffing capacities, create a stressful climate for nurses and other treatment facility staff. Suboptimal scheduling and complex treatment schedules can significantly increase the expenditures of cancer treatment facilities by requiring nursing staff to work long shifts, often beyond scheduled operating hours. Overtime and temporary labor expenses are a key concern for most treatment facilities. The effective management of a treatment facility depends mainly on optimizing patient scheduling and efficiently using available resources. With limited and localized information available while scheduling a patient for future treatment(s), it is unreasonable to expect the scheduler to evaluate different possibilities and perform efficient scheduling. As a result, the scheduler selects the future appointment time(s) taking into consideration limited amount of information, such as staff availability and patient preference, resulting in an unoptimized schedule for the day.

Hence, there is a need for an improved system and method for facilitating patient scheduling at a healthcare facility, in order to address the aforementioned issues.

### SUMMARY

This summary is provided to introduce a selection of concepts, in a simple manner, which is further described in the detailed description of the disclosure. This summary is neither intended to identify key or essential inventive concepts of the subject matter nor to determine the scope of the disclosure.

In accordance with an embodiment of the present disclosure, a computing system for facilitating patient scheduling at a healthcare facility is disclosed. The computing system includes one or more hardware processors and a memory coupled to the one or more hardware processors. The memory includes a plurality of modules in the form of programmable instructions executable by the one or more hardware processors. The plurality of modules include a request receiver module configured to receive a request from one or more electronic devices associated with a scheduler to schedule an appointment of a patient. The request includes but not limited to: a patient ID of the patient, a treatment profile of the patient, a treatment date and the like. The plurality of modules also include a data obtaining module configured to obtain at least one of: treatment information, provider information and resource information from an Electronic Health Record (EHR) system based on the received request. The treatment information includes the treatment profile and a treatment duration. The treatment duration is a time duration of the treatment profile. The data obtaining module obtains one or more available provider slots comprising MD or Nurse Practitioner (NP) time slots, from the EHR system based on the received request upon obtaining the at least one of: treatment information, provider information and resource information. Furthermore, the plurality of modules also include a time determination module configured to determine one or more optimal treatment times for the treatment date for the treatment profile of the patient based on the received request, the obtained at least one of: treatment information, provider information and resource information and the obtained one or more available slots. Furthermore, the plurality of modules includes a data output module configured to output the determined one or more optimal treatment times for the treatment date along with relevant patient information on user interface screen of the one or more electronic devices associated with the scheduler. The relevant patient information includes patient name, patient identifier, location of treatment, and date of the treatment.

In accordance with another embodiment of the present disclosure, a method for facilitating patient scheduling at a healthcare facility is disclosed. The method includes receiving a request from one or more electronic devices associated with a scheduler to schedule an appointment of a patient. The request includes but not limited to: a patient ID of the patient, a treatment profile of the patient, a treatment date and the like. The method further includes obtaining at least one of: treatment information, provider information and resource information from an Electronic Health Record (EHR) system based on the received request. The treatment information includes the treatment profile and a treatment duration. The treatment duration is a time duration of the treatment profile. Further, the method includes obtaining one or more available provider slots comprising MD or Nurse Practitioner (NP) time slots, from the EHR system based on the received request upon obtaining the at least one of: treatment information, provider information and resource information. Furthermore, the method includes determining one or more optimal treatment times for the treatment date for the treatment profile of the patient based on the received request, the obtained at least one of: treatment information, provider information and resource information and the obtained one or more available slots. The method includes outputting the determined one or more optimal treatment times for the treatment date along with relevant patient information on user interface screen of the one or more electronic devices associated with the scheduler. The relevant patient information includes patient name, patient identifier, location of treatment, and date of the treatment.

Embodiment of the present disclosure also provide a non-transitory computer-readable storage medium having instructions stored therein that, when executed by a hardware processor, cause the processor to perform method steps as described above.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF DRAWINGS

The disclosure will be described and explained with additional specificity and detail with the accompanying figures in which:

FIG. 1 is a block diagram illustrating an exemplary computing environment for facilitating patient scheduling at a healthcare facility, in accordance with an embodiment of the present disclosure;

FIG. 2 is a block diagram illustrating an exemplary computing system facilitating patient scheduling at the healthcare facility, in accordance with an embodiment of the present disclosure;

FIG. 3A-3B are graphical user interface screens of the computing system for facilitating patient scheduling at the healthcare facility, in accordance with an embodiment of the present disclosure;

FIG. 4 is a block diagram depicting static optimized Day of Week (DOW) template profiles being disassembled and stored as time stamped service types, in accordance with an embodiment of the present disclosure;

FIG. 5 is a tabular representation depicting disassembled time stamped service types derived from static optimized DOW template, in accordance with an embodiment of the present disclosure;

FIG. 6 is a block diagram depicting combining of dynamically matched profiles with dynamic EHR data, in accordance with an embodiment of the present disclosure;

FIG. 7 is an exemplary block diagram depicting suggested optimized times by the computing system, in accordance with an embodiment of the present disclosure;

FIG. 8 is a process flow diagram illustrating an exemplary method for facilitating patient scheduling at the healthcare facility, in accordance with an embodiment of the present disclosure;

FIG. 9 depicts a process flow diagram in accordance with some embodiments of the present disclosure; and

FIG. 10 depicts a process flow diagram in accordance with some embodiments of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION OF THE DISCLOSURE

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure. It will be understood by those skilled in the art that the foregoing general description and the following detailed description are exemplary and explanatory of the disclosure and are not intended to be restrictive thereof.

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

The terms “comprise,” “comprising,” or any other variations thereof, are intended to cover a non-exclusive inclusion, such that one or more devices or sub-systems or elements or structures or components preceded by “comprises . . . a” does not, without more constraints, preclude the existence of other devices, sub-systems, additional sub-modules. Appearances of the phrase “in an embodiment,” “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

A computer system (standalone, client or server computer system) configured by an application may constitute a “module” (or “subsystem”) that is configured and operated to perform certain operations. In one embodiment, the “module” or “subsystem” may be implemented mechanically or electronically, so a module include dedicated circuitry or logic that is permanently configured (within a special-purpose processor) to perform certain operations. In another embodiment, a “module” or “subsystem” may also comprise programmable logic or circuitry (as encompassed within a general-purpose processor or other programmable processor) that is temporarily configured by software to perform certain operations.

Accordingly, the term “module” or “subsystem” should be understood to encompass a tangible entity, be that an entity that is physically constructed permanently configured (hardwired) or temporarily configured (programmed) to operate in a certain manner and/or to perform certain operations described herein.

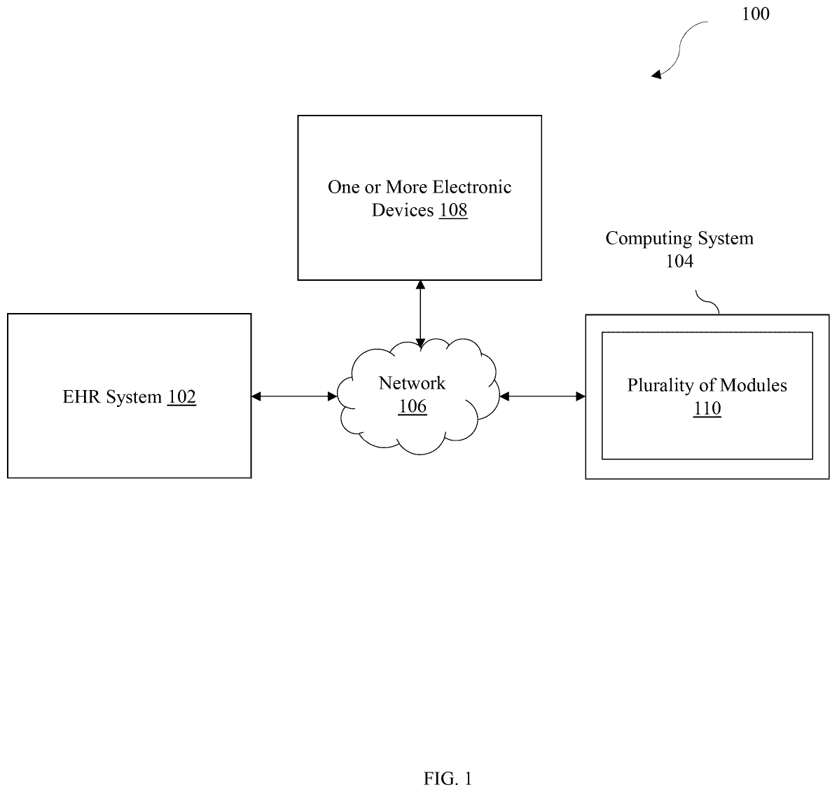

Referring now to the drawings, and more particularly to FIG. 1 through FIG. 8, where similar reference characters denote corresponding features consistently throughout the figures, there are shown preferred embodiments and these embodiments are described in the context of the following exemplary system and/or method.

FIG. 1 is a block diagram illustrating an exemplary computing environment 100 facilitating patient scheduling at a healthcare facility, in accordance with an embodiment of the present disclosure. According to FIG. 1 the computing environment 100 includes an Electronic Health Record (EHR) system 102 communicatively coupled to a computing system 104 via a network 106. In an embodiment of the present disclosure, the EHR system 102 is an external database for storing treatment information, provider information, resource information, or any combination thereof. Further, the network 106 may be internet or any other wireless network. The computing system 104 may be hosted on a central server, such as cloud server or a remote server.

Further, the computing environment 100 includes one or more electronic devices 108 associated with a scheduler communicatively coupled to the computing system 104 via the network 106. In an embodiment of the present disclosure, the scheduler is a user who schedules appointment of one or more patients at a healthcare facility. In an exemplary embodiment of the present disclosure, the healthcare facility includes ambulatory surgical centers, blood banks, clinics and medical offices, dialysis centers, hospice homes, hospitals, imaging, and radiology centers, and the like. In an embodiment of the present disclosure, the one or more electronic devices 108 are configured to receive the request from the one or more electronic devices 108 associated with the scheduler to schedule an appointment of the patient. The one or more electronic devices 108 also provide one or more optimal treatment times for a treatment date along with relevant patient information to the computing system 104. In an exemplary embodiment of the present disclosure, the one or more electronic devices 108 may include a laptop computer, desktop computer, tablet computer, smartphone, wearable device, a digital camera and the like.

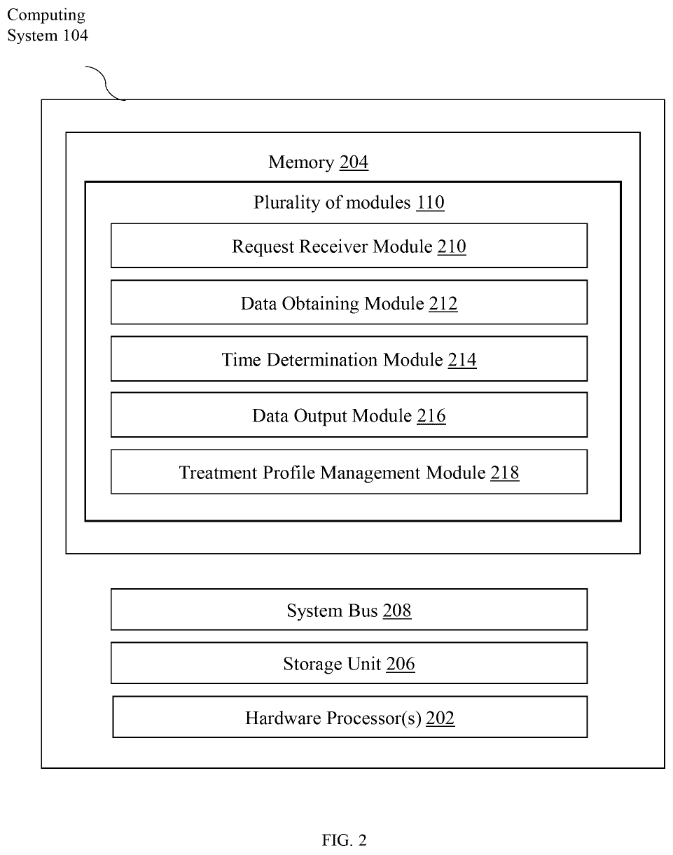

Furthermore, the one or more electronic devices 108 include a local browser, a mobile application, or a combination thereof. Furthermore, the scheduler may use a web application via the local browser, the mobile application, or a combination thereof to communicate with the computing system 104. In an exemplary embodiment of the present disclosure, the mobile application may be compatible with any mobile operating system, such as android, IOS, and the like. In an embodiment of the present disclosure, the computing system 104 includes a plurality of modules 110. Details on the plurality of modules 110 have been elaborated in subsequent paragraphs of the present description with reference to FIG. 2.

In an embodiment of the present disclosure, the computing system 104 is configured to receive a request from the one or more electronic devices 108 associated with the scheduler to schedule an appointment of a patient for the treatment profile. Further, the computing system 104 obtains the treatment information, provider information and resource information from the EHR system 102 based on the received request. Furthermore, the computing system 104 obtains the one or more available slots of provider slots comprising MD or Nurse Practitioner (NP) time slots, from the EHR system 102 based on the received request upon obtaining the treatment information, provider information and resource information. The computing system 104 determines one or more optimal treatment times for the treatment date and alternate treatment date for the treatment profile of the patient based on the received request, the treatment information, the provider information, the resource information and the obtained one or more available slots. Further, the computing system 104 outputs the determined one or more optimal treatment times for the treatment date and alternate treatment date along with the relevant patient information on user interface screen of the one or more electronic devices 108 associated with the scheduler.

FIG. 2 is a block diagram illustrating an exemplary computing system 104 facilitating patient scheduling at the healthcare facility, in accordance with an embodiment of the present disclosure. Further, the computing system 104 includes one or more hardware processors 202, a memory 204 and a storage unit 206. The one or more hardware processors 202, the memory 204 and the storage unit 206 are communicatively coupled through a system bus 208 or any similar mechanism. The memory 204 comprises the plurality of modules 110 in the form of programmable instructions executable by the one or more hardware processors 202. Further, the plurality of modules 110 includes a request receiver module 210, a data obtaining module 212, a time determination module 214, a data output module 216 and a treatment profile management module 218.

The one or more hardware processors 202, as used herein, means any type of computational circuit, such as, but not limited to, a microprocessor unit, microcontroller, complex instruction set computing microprocessor unit, reduced instruction set computing microprocessor unit, very long instruction word microprocessor unit, explicitly parallel instruction computing microprocessor unit, graphics processing unit, digital signal processing unit, or any other type of processing circuit. The one or more hardware processors 202 may also include embedded controllers, such as generic or programmable logic devices or arrays, application specific integrated circuits, single-chip computers, and the like.

The memory 204 may be non-transitory volatile memory and non-volatile memory. The memory 204 may be coupled for communication with the one or more hardware processors 202, such as being a computer-readable storage medium. The one or more hardware processors 202 may execute machine-readable instructions and/or source code stored in the memory 204. A variety of machine-readable instructions may be stored in and accessed from the memory 204. The memory 204 may include any suitable elements for storing data and machine-readable instructions, such as read only memory, random access memory, erasable programmable read only memory, electrically erasable programmable read only memory, a hard drive, a removable media drive for handling compact disks, digital video disks, diskettes, magnetic tape cartridges, memory cards, and the like. In the present embodiment, the memory 204 includes the plurality of modules 110 stored in the form of machine-readable instructions on any of the above-mentioned storage media and may be in communication with and executed by the one or more hardware processors 202.

In an embodiment of the present disclosure, the storage unit 206 may be a cloud storage. The storage unit 206 may store the received request, the treatment information, the provider information, the resource information, the one or more treatment dates, one or more alternate treatment dates, the one or more optimal treatment times for each of the one or more treatment dates, the one or more optimal treatment times for each of the one or more alternate treatment dates, one or more exact matches, a set of static optimized Day of the Week (DOW) templates for each DOW, one or more approximate matches, a set of optimized and prioritized profiles, a set of rank ordered time slots, optimized schedules and the like.

The request receiver module 210 is configured to receive the request from the one or more electronic devices 108 associated with the scheduler to schedule the appointment of the patient for the treatment profile. For example, the request includes but not limited to: a patient ID of the patient, a treatment profile of the patient, a treatment date, alternate treatment date and the like. In an exemplary embodiment of the present disclosure, the treatment profile includes one or more lab tests, services to be scheduled with a planned duration for each of one or more medical services, an order in which the one or more medical services are required to be scheduled, an appointment with Medical Assistant (MA), Patient Medical Record (MRN) number, one or more different medical services that are part of the treatment profile, appointment with a physician or a nurse practitioner (MD), and the like. For example, the one or more medical services include injection, treatment, lab tests to a patient, and the like.

The data obtaining module 212 obtains the treatment information, the provider information, the resource information, or any combination thereof from the EHR system 102 based on the received request. In an embodiment of the present disclosure, the treatment information includes the treatment profile and a treatment duration. In an embodiment of the present disclosure, the treatment duration is a time duration of the treatment profile. In an exemplary embodiment of the present disclosure, the provider information includes provider name, provider location, provider skillset, provider schedule, provider availability, and the like. In an embodiment of the present disclosure, the provider is a person or a set of persons performing the one or more medical services. For example, the provider includes a physician, a group of physicians, clinic, facility that is part of a hospital or a health system, and one or more other persons or an entity that provides treatment to patients. In an exemplary embodiment of the present disclosure, the resource information includes one or more resources where each of one or more medical services are required to be scheduled, current utilization and availability of each of the one or more resources. For example, the one or more resources include lab chair, treatment chair, hospital stretcher, defibrillators used as part of patient treatment procedure, or any combination thereof.

Further, the data obtaining module 212 obtains the one or more available provider slots comprising MD or Nurse Practitioner (NP) time slots, from the EHR system 102 based on the received request upon obtaining the treatment information, the provider information, the resource information, or any combination thereof. For example, the one or more available slots may be from 4:30 PM to 5:00 PM, 6:00 PM to 6:30 PM, and the like. In an embodiment of the present disclosure, the treatment information, the provider information, the resource information, and the one or more available slots are obtained in real-time.

The time determination module 214 is configured to determine the one or more optimal treatment times for the treatment date and alternate treatment date for the treatment profile of the patient based on the received request, the obtained treatment information, the obtained provider information, the obtained resource information, or any combination thereof, and the obtained one or more available slots. In an embodiment of the present disclosure, the time determination module 214 also determines one or more treatment dates and the one or more optimal treatment times for each of the one or more treatment dates for the treatment profile of the patient based on the received request, the obtained treatment information, the obtained provider information, the obtained resource information, or any combination thereof, and the obtained one or more available slots.

The time determination module 214, determines the one or more treatment times by implementing the following steps. In the first step, the patient visit date and alternate visit date is provided by the physician using the EHR. In the second step, the scheduler utilizes the above visit date and alternate visit date to select specific timings depending on the different services requested for the visit. The services included in the visit can be any combination of Lab, MA, MD, treatment, and/or injection, treatment can be one of several durations ranging from 15 mins to 8 hours or more in increments of 15 mins. In the third step, the scheduler is assisted in selecting the specific timings based on available resources for the services included in the visit. For example, in case a patient needs to come in for a 15 min MD visit and 90 min treatment for a day in the future. Then for that specific day, the required MD is available at 11 AM, 11:30 AM, and 2 PM and the treatment room has capacity to treat the patient any time between 11 PM and 4 PM. Further, patient visit date, services included in the visit, MD availability, and treatment room availability are sent in accordance with the present disclosure. Furthermore, based on the aforementioned details and the static DOW template, the systems and methods disclosed herein identify that 2 PM MD visit and 2:15 PM treatment is the optimal time, thereby presenting this output to the scheduler. Additionally, the scheduler utilizes the above time to schedule the visit in the EHR.

In an embodiment of the present disclosure, the time determination module 214 is configured to determine one or more exact matches or one or more approximate matches corresponding to the one or more optimal treatment times based on the received request, the obtained treatment information, the obtained provider information, the obtained resource information, or any combination thereof, and the obtained one or more available slots. In an embodiment of the present disclosure, the one or more exact matches are the one or more appointment times which exactly correspond to the treatment profile under consideration. Further, the one or more approximate matches are the one or more appointment times which correspond in an approximate manner to the treatment profile under consideration. In an embodiment of the present disclosure, the one or more appointment times may be displayed in a specific way to differentiate the one or more exact matches from the one or more approximate matches.

The time determination module 214 determines the one or more exact matches or one or more approximate matches by implementing the following steps. In the first step, the patient visit date is provided by the physician using the EHR. In the second step, the scheduler utilizes the above visit date to select a specific time depending on the different services requested for the visit. The services included in the visit comprises a combination of Lab, MA, MD, treatment, and/or Injection, treatment can be one of several durations ranging from 15 mins to 8 hours or more in increments of 15 mins. In the third step, the present disclosure assists the scheduler in selecting the time based on available resources for the services included in the visit. For example, in case a patient needs to come in for a 15 min MD visit and 90 min treatment for a day in the future. Then it is noted that for that specific day, the required MD is available at 11 AM, 11:30 AM, and 2 PM and the treatment room has a capacity to treat the patient any time between 11 PM and 4 PM. Further, the patient visit date, services included in the visit, MD availability, and treatment room availability are sent to the present disclosure. Furthermore, the present disclosure's DOW template may include 2 PM MD visit and 2:15 PM 90 treatments available in it. The DOW template may also include 11 AM MD visit with 120 min treatment at 11:15 AM. Based on the visit details and the present disclosure's static DOW template, 2 PM MD visit and 2:15 PM treatment visit will be shown as an exact match and 11 AM MD visit with 11:15 AM treatment will be shown as an approximate match since the treatment duration for this time is 120 mins in the template and not 90 mins but still a treatment can be scheduled. Additionally, the scheduler will use the above presented times by the present disclosure to schedule the visit in the EHR.

The data output module 216 is configured to output the determined one or more optimal treatment times for the treatment date and alternate treatment date along with the relevant patient information on user interface screen of the one or more electronic devices 108 associated with the scheduler. In an exemplary embodiment of the present disclosure, the relevant patient information includes patient name, patient identifier, location of treatment, date of the treatment, and the like. In an exemplary embodiment of the present disclosure, the one or more electronic devices 108 may include a laptop computer, desktop computer, tablet computer, smartphone, wearable device, a digital camera and the like. In an embodiment of the present disclosure, the scheduler may use the determined one or more optimal treatment times to schedule the patient's treatment.

In a use-case scenario, a scheduler may want to schedule patient ‘P’ for treatment profile ‘Tx’ on date ‘D’ in the future. In order to obtain optimal treatment times to schedule Tx on date D, the scheduler may use a trigger, such as, but not limited to a button click in a software program. This trigger may then initiate the process of obtaining all the relevant information such as, but not limited to, patient MRN, different services that are part of Tx, staff information, staff schedule, and the like from the EHR system 102. This information is then sent to the computing system 104. Response from the computing system 104 may then be received as a list of optimal appointment times to schedule treatment profile Tx for patient P on date D. This information may then be presented to the end-user in a graphical user interface as part of a software program.

In another use-case scenario, the scheduler may want to schedule patient ‘P’ for treatment profile ‘Tx’ on date ‘D’ in the future. In order to obtain optimal treatment times to schedule Tx on date D, the required information may be obtained from the EHR and sent to the computing system 104 that responds with optimal start times for Tx. The computing system 104 may respond with the one or more exact matches and the one or more approximate matches. The received appointment times may be displayed in a specific way to differentiate exact matches from approximate matches. For examples, exact matches may be displayed in bold font while approximate matches are displayed in normal font.

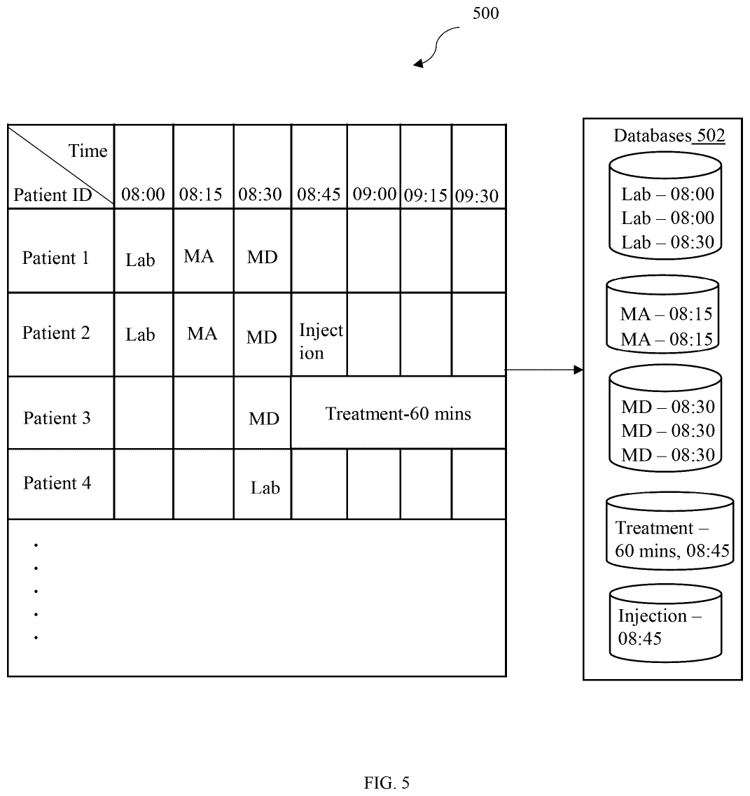

In yet another use-case scenario, the scheduler may want to schedule patient ‘P’ for treatment profile ‘Tx’ on date ‘D’ in the future. The treatment profile may include additional information that requires special handling. For example, patient P may be a new patient undergoing treatment for the first time and requires 15 minutes MD new patient appointment. In order to obtain optimal treatment times for this treatment profile, Tx on date D, the required information may be obtained from the EHR system 102 and sent to the computing system 104 that responds with optimal start times for Tx. The computing system 104 may respond with exact matches that identify MD availability to see new patients. The computing system 104 may also provide approximate matches where the MD is available, but the available slots may not be specific to new patients. The received appointment times may be displayed in a specific way to differentiate MD new patient slots from MD slots not specific to new patients. For example, MD new patient blocks may be highlighted using options such as, but not limited to, different background color, different font type, different font color, and the like. In an embodiment of the present disclosure, the treatment profile management module 218 receives historical patient data associated with a patient from the EHR system 102. In an exemplary embodiment of the present disclosure, the historical patient data include service date, a breakdown of different services needed for each treatment, staff schedules, operating hours of the provider, and the like. Further, the treatment profile management module 218 generates the set of static optimized Day of the Week (DOW) templates for each DOW based on the received historic patient data by performing a statistical and combinatorial optimization analysis on the received historical patient data. In an embodiment of the present disclosure, the set of static optimized DOW templates include forecasted patient profiles assigned to optimized time slots. The forecasted patient profiles correspond to various service type combinations. The DOW template is generated based on historic data by implementing the following steps. In the first step, the historical patient visits provide details including but not limited to the number of visits scheduled for a given day, the types of visits including any combination of Lab, MA, MD, treatment, and/or injection and treatment duration with treatments requiring 15 mins to more than 8 hours in 15 min increments. Further, the historical patient visits also provide details regarding the distribution pattern of different types of visits. For example, the percentage of total visits with injection only requirement, the percentage of total visits with MD and treatment requirement and the like. In the second step, the statistical models are utilized on the above historical data to identify reliable patterns. The patterns seem to show consistency across different days of the week. For example, most Mondays typically seem to have similar number of visits and percentage distribution between different types of visits. Similarly, Tuesday, Wednesdays, and the like. In the third step, predictive modeling techniques are used to project the aforementioned observations into the future to come up with patterns for each day of the week and different visits are accommodated into each DOW template based on these predictions. For example, for Mondays-Lab at 7:30 AM followed by MD visit at 7:45 AM followed by 360 min treatment at 8 AM; Lab starting at 10 AM followed by 240 min treatment at 8:15 AM; MD visit at 10 AM followed by 90 min treatment at 10:15 AM followed by injection at 10:30 AM and the like. The treatment profile management module 218 classifies the generated set of static optimized DOW templates into various service types. FIG. 5 illustrates a non-limiting example of this approach, as discussed herein, utilized by the treatment profile management module 218 to classify the generated set of optimized DOW templates into various service types. For example, according to some embodiments, FIG. 5 depicts a DOW template with four patient profiles assigned to specific time slots. Patient 1 has Lab, MA, MD with Lab starting at 08:00, MA starting at 08:15 and MD starting at 08:30. Patient 2 has Lab, MA, MD, Injection with Lab starting at 08:00, MA starting at 08:15, MD starting at 08:30 and Injection starting at 08:45. The present disclosure utilizes these two patient profiles in the DOW template to illustrate the method used in the classification of DOW templates into various time-stamped service types. The present disclosure first creates different service buckets for each service type, namely, Lab, MA, MD, Injection and Treatment. From the profile of patient 1, the present disclosure determines that there is one lab service starting at 08:00, one MA service starting at 08:15 and one MD service starting at 08:30. The present disclosure then creates a time stamped entry corresponding to Lab-08:00 and puts it into the Lab service bucket. Similarly, it creates and enters MA-08:15 into the MA service bucket and MD-08:30 into the MD service bucket as illustrated in FIG. 5. Further, it looks at the profile for patient 2 and creates and enters the following four service types into their corresponding buckets: Lab-08:00, MA-08:15, MD-08:30 and Injection-08:45 as shown in FIG. 5. The present disclosure continues to go down the list and examines other profiles within the DOW template and classifies the constituent service types into time-stamped service types and places the time-stamped service types in the appropriate service bucket, as shown in FIG. 5. The treatment profile management module 218 categorizes the forecasted patient profiles into individual time stamped services upon classifying the generated set of static optimized DOW templates. In an embodiment of the present disclosure, the forecasted patient profiles are disassembled. A patient profile corresponds to a combination of one or more services such as lab, MA, MD, treatment, injection and the like. Statistically optimized static DOW template has different profiles assigned to various times of the day. For example, Profile1=90-minute treatment profile starting at 10:30 AM (assigned to 10:30 AM). Profile2=15 min lab+15 min MD+60 min treatment profile assigned to 1 PM. Which means lab starts at 1 PM, MD starts at 1:15 PM and treatment starts at 1:30 PM. Profile3=15 min lab+15 min MD+90 min treatment assigned to 1 PM. implying that the lab starts at 1 PM, MD starts at 1:15 PM and injection starts at 1:30 PM. The profiles from the static DOW template are broken down into their individual services and stored along with their respective timestamps Therefore, based on the aforementioned examples on the profiles being disassembled and timestamped, there exists: a one 90 min treatment at 10:30 AM (from profile1), two labs at 1 PM (from profile2 and profile3), two MD at 1:15 PM (from profile2 and profile3), one 60 min treatment at 1:30 PM (from profile2), one 90 min treatment at 1:30 PM (from profile3) and the like. Furthermore, the treatment profile management module 218 generates a set of optimized and prioritized profiles based on time stamped individual service type, dynamic EHR data, and a profile of the patient to be currently scheduled upon categorizing the forecasted patient profiles. In case a 90-minute treatment is requested for a patient, there are two times available, based on the aforementioned example—10:30 AM from profile1 and 1:30 PM from profile3. Based on the real time data obtained from the EHR, if there is already one treatment starting at 10:30 AM, but there are not treatments starting at 1:30 PM, then the 1:30 PM time is prioritized in order to evenly balance the load across the day. Hence 1:30 PM will be provided as the first option and 10:30 AM as the second option, thereby creating a prioritized list based on the timestamped services obtained from the static DOW template, real time EHR data and requested patient visit.

In an embodiment of the present disclosure, the EHR data includes specific details about already scheduled assignments. The dynamically assembled matched profiles combines with the EHR data to generate the set of optimized and prioritized profiles. The treatment profile management module 218 determines a set of rank ordered time slots based on the dynamic EHR data, the static optimized DOW template, one or more patient's preferences and one or more different resources required by a specific patient profile by using a patient scheduling-based Artificial Intelligence (AI) model. It is possible that the static DOW template has multiple matching times for a requested patient visit. In order to present the multiple matching times in a list, the multiple matching times are ranked in the order of most optimal time to least optimal time. The ranking is based on the available times from DOW template, already scheduled patients for those time obtained from dynamic EHR data and the like. Therefore, the set of rank ordered slots corresponds to the ranking of the multiple matching times. In an exemplary embodiment of the present disclosure, the one or more patient's preferences include a preferred date, a preferred time, a preferred medical professional, and the like. Further, the treatment profile management module 218 outputs the determined set of rank ordered time slots and the generated set of optimized and prioritized profiles on user interface screen of the one or more electronic devices 108 associated with the scheduler.

In determining the set of rank ordered time slots based on the dynamic EHR data, the static optimized DOW template and the one or more different resources required by the specific patient profile by using the patient scheduling-based AI model, the treatment profile management module 218 correlates information pertaining to the time slots assigned to various service types derived from the static DOW template with specifics of the dynamic EHR data for a selected clinic for a selected treatment date and alternate treatment date by using the patient scheduling-based AI model. Further, the treatment profile management module 218 determines the set of rank ordered time slots based on result of correlation.

For example, the computing system 104 dynamically assembles profiles to match the selected patient profile in order to identify most optimal future appointment times for patient treatments to be scheduled at healthcare facilities. The computing system 104 stores information derived from a static optimization of forecasted patient profiles for a specific day of a week, known by those familiar with the art as a day of the week (DOW) template, in the form of time stamped service types, and at the time of scheduling, allows the stored information to be retrieved in real time to dynamically assemble a profile matching the selected patient profile. This may further be combined with actual schedule information obtained from the EHR system 102 for a specific future date, in an intelligent fashion to produce prioritized optimized schedules for specific treatment profiles. Staff schedule includes but is not limited to, a detailed listing of the availability of doctors, nurse practitioners, nurses, lab technicians, medical assistants, and the like. Treatment or treatment profile or patient profile refers to any combination of different services such as, but not limited to, lab tests, appointment with medical assistant (MA), appointment with a physician or a nurse practitioner (MD), application of injection, providing treatment and the like. Provider can be, but not limited to a physician, group of physicians, clinic, facility that is part of a hospital or a health system, or any other person(s) or an entity that provides treatment to patients. Staff refers to person or persons performing services such as, but not limited to, injection, treatment, lab tests and the like to a patient. Resource refers to equipment such as, but not limited to, lab chair, treatment chair and the like utilized as part of patient treatment procedure.

In an embodiment of the present disclosure, the treatment profile management module 218 obtains one or more inputs from the EHR system 102. In an exemplary embodiment of the present disclosure, the one or more inputs include patient MRN, treatment schedule date, treatment schedule location, one or more resources, availability of the one or more resources, resource duration, provider schedule, number of patients assigned to each time slot, one or more medical services required by each patient, or any combination thereof. Further, the treatment profile management module 218 obtains an input from the statically optimized DOW template.

Furthermore, the treatment profile management module 218 determines optimized schedules for specific patient profile under consideration based on the obtained one or more inputs from the EHR system 102 and the obtained input from the statically optimized DOW template. Optimized schedules correspond to one or more specific time slots of the day for the given day of the week when the set of requested services can be scheduled in an efficient way. Further, the treatment profile management module 218 determines optimized schedules by implementing the following steps. In the first step, the historical patient visits provide details including but not limited to the number of visits scheduled for a given day, the types of visits including any combination of Lab, MA, MD, treatment, and/or injection and treatment duration with treatments requiring 15 mins to more than 8 hours in 15 min increments. Further, the historical patient visits also provide details regarding the distribution pattern of different types of visits. For example, the percentage of total visits with injection only requirement, the percentage of total visits with MD and treatment requirement and the like. In the second step, the statistical models are utilized on the above historical data to identify reliable patterns. The patterns seem to show consistency across different days of the week. For example, most Mondays typically seem to have similar number of visits and percentage distribution between different types of visits. Similarly, Tuesday, Wednesdays, and the like. In the third step, predictive modeling techniques are used to project the aforementioned observations into the future to come up with patterns for each day of the week and different visits are accommodated into each DOW template based on these predictions. For example, for Mondays—Lab at 7:30 AM followed by MD visit at 7:45 AM followed by 360 min treatment at 8 AM; Lab starting at 10 AM followed by 240 min treatment at 8:15 AM; MD visit at 10 AM followed by 90 min treatment at 10:15 AM followed by injection at 10:30 AM and the like.

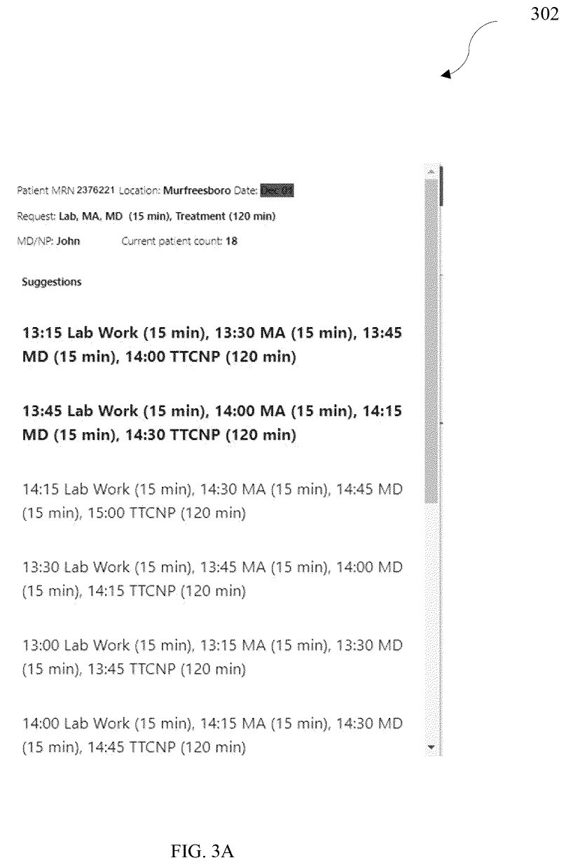

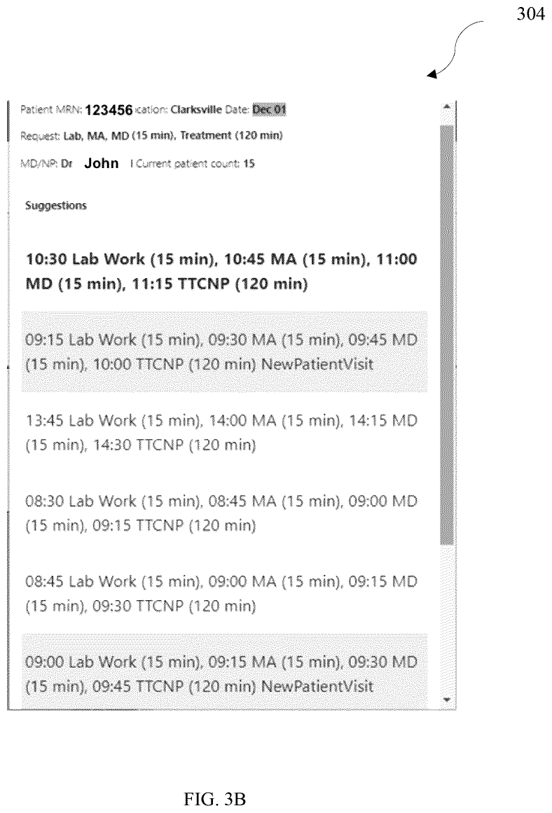

FIG. 3A-3B are graphical user interface screens of the computing system 104 for facilitating patient scheduling at the healthcare facility, in accordance with an embodiment of the present disclosure. The graphical user interface screen 302 of FIG. 3A depicts an example where the optimal treatment times received from the computing system 104 are presented to the end user as part of a computer software program. In this example, exact matches to the user requested treatment profile are highlighted in bold and prioritized along with approximate matches that are displayed in normal font. Further, the graphical user interface screen 304 of FIG. 3B depicts where the treatment times received from the computing system 104 are presented to the end user as part of a computer software program. In this example, exact matches to the user requested treatment profile are highlighted in bold and prioritized along with approximate matches that are displayed in normal font. Furthermore, new patient blocks are also highlighted.

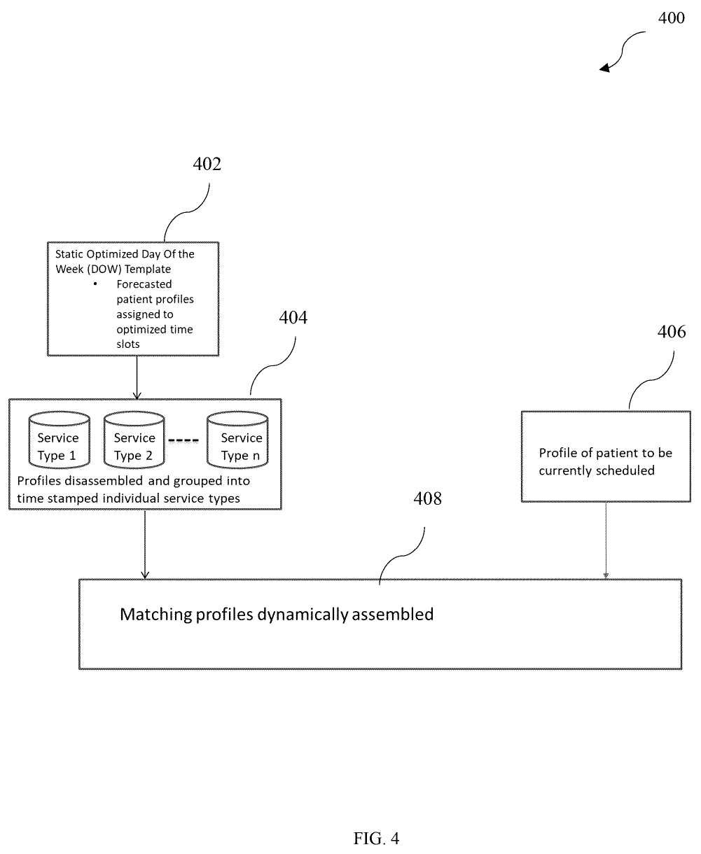

FIG. 4 is a block diagram 400 depicting static optimized DOW template profiles being disassembled and stored as time stamped service types, in accordance with an embodiment of the present disclosure. At step 402, a static optimized DOW template includes forecasted patient profiles assigned to optimized time slots. The static optimized DOW template is classified into various service types. At step 404, profiles are disassembled and grouped into time stamped individual service types. At step 406, profile of patient to be currently scheduled is included. At step 408, the time stamped individual service types are retrieved and dynamically assembled to produce profiles matching a selected profile of patient to be currently scheduled.

FIG. 5 is a tabular representation 500 depicting disassembled time stamped service types derived from static optimized DOW template, in accordance with an embodiment of the present disclosure. Here, the disassembling of the DOW profiles and storage of resulting service types are depicted. A static optimized DOW templates are derived from historic patient data through a variety of statistical and combinatorial optimization analysis. The net result of such a process is DOW template for each DOW. An illustrative DOW template with four patient profiles is depicted in the tabular representation 500. Here, patient 1 has to go to a lab for fifteen minutes, requires Medical Assistant (MA) for fifteen minutes and has an appointment with a physician or a nurse practitioner (MD) for fifteen minutes in sequential order starting at 08:00 and going up to 08:45. Further, patient 2 has to go to the lab for fifteen minutes, requires MA for fifteen minutes, has appointment with a physician or an MD for fifteen minutes and requires application of an injection for fifteen minutes in a sequential order starting at 08:00 and going up to 09:00. Further, patient 3 has appointment with a physician or a MD for fifteen minutes starting at 08:30 followed by a treatment for sixty minutes going up to 09:30. Further, patient 4 has to go to the lab for fifteen minutes starting at 8:30. The present disclosure disassembles the component service type of each profile in the DOW template, time stamps it to indicate the starting time for that service type and stores it in a unique database for each service type. Here, five databases 502 are depicted, one for each service type, populated with time stamped service types derived from the DOW template. A lab service type database has three-time stamped lab service types two starting at 08:00 derived from the profiles of the patient 1 and the patient 2, and a third lab service type starting at 08:30 derived from the profile of the patient 4. The other service type databases are populated in a similar manner with the starting times of service types derived from the patient profiles in the DOW template. During the scheduling process, at the time when a patient with a specific profile is presented, a matching profile is dynamically assembled in real time by piecing together individual service types stored within service type databases.

In a preferred embodiment, the patient profile may comprise one or more services. The static optimized DOW template for the schedule date corresponding to this patient profile may contain an ordered list of times corresponding to exact matches and approximate matches. The exact matches are those that correspond exactly to the specific patient profile under consideration. Approximate matches are those that correspond in an approximate manner to the specific patient profile under consideration.

In an exemplary embodiment, a patient profile (Tx1) may comprise fifteen minutes lab, fifteen minutes MA requirement and fifteen minutes appointment with a physician or a MD. This patient may need to be scheduled on a day whose template is shown in FIG. 5. For this treatment, the present disclosure dynamically assembles two matching profiles by piecing together lab starting at 08:00, requirement of MA starting at 08:15 and appointment with a physician or a MD starting at 08:30. This leads to two lab, requirement of MA and appointment with a physician or a MD matching profile both starting at 08:00. A second patient profile (Tx2) may comprise fifteen minutes lab and sixty minutes treatment. For this patient, the present disclosure assembles a matching profile by piecing together the fifteen-minute lab starting at 08:30 and a sixty-minute treatment starting at 08:45. In both these instances there is an exact match between the patient profile and the profile assembled by the present disclosure. Consider a third example where patient profile (Tx3) is considered that comprises fifteen minutes lab and thirty minutes treatment. In this case, the present disclosure assembles a profile with fifteen minutes lab starting at 08:30 and thirty-minute treatment starting at 08:45 resulting in an approximate match. The match is approximate as the duration of the treatment starting at 08:45 within the template is sixty minutes which is different from the treatment duration of the patient profile (Tx3) which is thirty minutes. Additionally, the present disclosure may allow the scheduler to manually select any desired time slot or time slots for a specific treatment profile. The manual option may be used by the scheduler either when no exact matches or approximate matches are available, or the scheduler wishes to make a selection other than the recommended optimized time slot or time slots.

In another embodiment, it is inferred that, input from an EHR system 102 such as, but not limited to, patient MRN, treatment schedule date, treatment schedule location, resources needed (such as lab chair, treatment chair and the like.), resources available (such as lab chair, treatment chair, and the like), resource duration, staff schedule (such as availability of medical assistant, nurse and the like), the number of patients assigned to each time slot and the services they require, is combined with input from a statically optimized DOW template to derive dynamically optimized schedules for the specific patient profile under consideration.

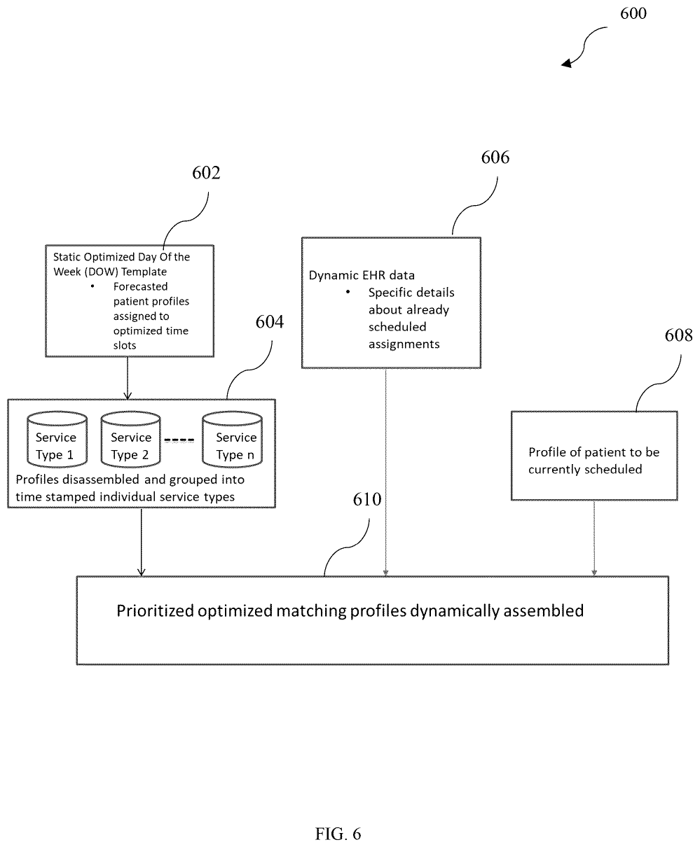

FIG. 6 is a block diagram 600 depicting combining of dynamically matched profiles with dynamic EHR data, in accordance with an embodiment of the present disclosure. At step 602, a static optimized DOW template comprises forecasted patient profiles assigned to optimized time slots. The static optimized DOW template is classified into various service types. At step 604, profiles are disassembled and grouped into individual time stamped service types. At step 606, the dynamic EHR data comprises specific details about already scheduled assignments. At step 608, profile of patient to be currently scheduled are included. At step 610, the time stamped individual service type, the dynamic EHR and profile of the patient to be currently scheduled may be utilized to generate optimized, prioritized list of profiles. Further, the dynamically assembled matched profiles combines with the dynamic EHR data to generate optimized and prioritized list of profiles. A scheduling algorithm takes different resources into consideration required by the specific patient profile and recommends rank ordered time slots based on the dynamic EHR data and the static optimized DOW template. The scheduling algorithm achieves this by combining information pertaining to the time slots assigned to various service types derived from the static DOW template with the specifics of the EHR data for the chosen clinic for the chosen day.

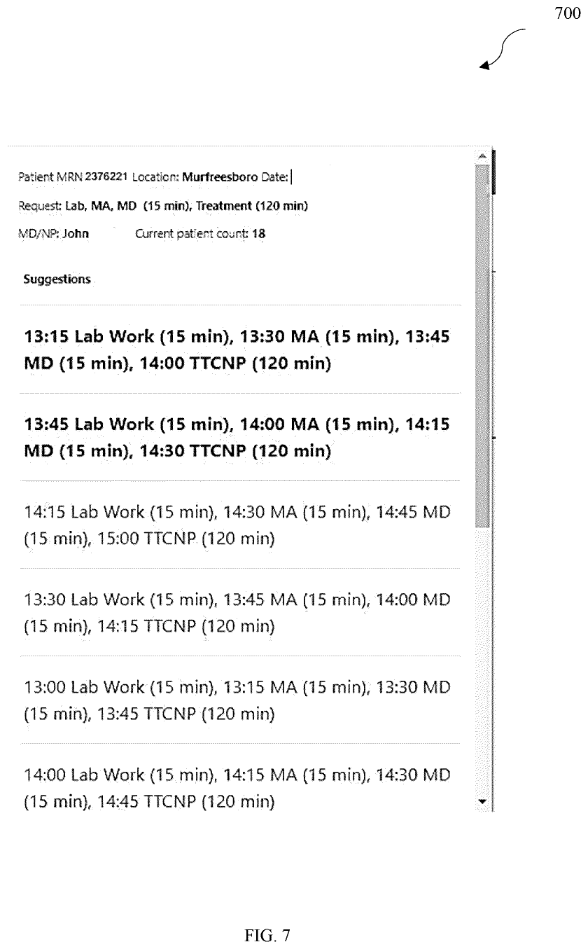

FIG. 7 is an exemplary block diagram 700 depicting suggested optimized times by the computing system 104, in accordance with an embodiment of the present disclosure. The suggested optimized time by the computing system 104 is presented to an end user as part of a computer software program. In this example, exact matches between user requested treatment profile and DOW template suggested treatment profile are highlighted in bold and prioritized along with approximate matches. A patient profile may comprise one or more services. Static optimized DOW template for this patient profile may provide many possible start times of T1, T2 and the like. This recommended set of start times in conjunction with information obtained from the EHR as described above may result in the algorithm prioritizing the time slots T1, T2 and the like into an ordered list Ti1, Ti2 and the like. This prioritized list is then presented to the end user as part of a computer program.

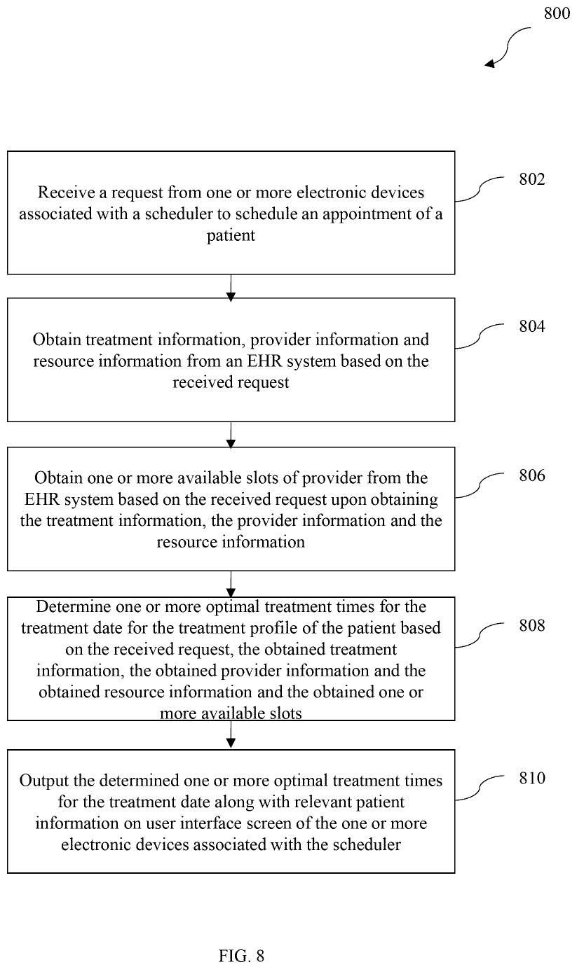

FIG. 8 is a process flow diagram illustrating an exemplary method for facilitating patient scheduling at the healthcare facility, in accordance with an embodiment of the present disclosure. At step 802, a request is received from one or more electronic devices 108 associated with a scheduler to schedule an appointment of a patient for a treatment profile. For example, the request includes a patient ID of the patient, a treatment profile of the patient, a treatment date, an alternate treatment date and the like. In an exemplary embodiment of the present disclosure, the treatment profile includes one or more lab tests, services to be scheduled with a planned duration for each of one or more medical services, an order in which the one or more medical services are required to be scheduled, an appointment with Medical Assistant (MA), Patient Medical Record (MRN) number, one or more different medical services that are part of the treatment profile, appointment with a physician or a nurse practitioner (MD), and the like. For example, the one or more medical services include injection, treatment, lab tests to a patient, and the like.

At step 804, treatment information, provider information, resource information, or any combination thereof are received from an EHR system 102 based on the received request. In an embodiment of the present disclosure, the treatment information includes the treatment profile and a treatment duration. In an embodiment of the present disclosure, the treatment duration is a time duration of the treatment profile. In an exemplary embodiment of the present disclosure, the provider information includes provider name, provider location, provider skillset, provider schedule, provider availability, and the like. In an embodiment of the present disclosure, the provider is a person or a set of persons performing the one or more medical services. For example, the provider includes a physician, a group of physicians, clinic, facility that is part of a hospital or a health system, and one or more other persons or an entity that provides treatment to patients. In an exemplary embodiment of the present disclosure, the resource information includes one or more resources where each of one or more medical services are required to be scheduled, current utilization and availability of each of the one or more resources. For example, the one or more resources include lab chair, treatment chair, hospital stretcher, defibrillators used as part of patient treatment procedure, or any combination thereof.

At step 806, one or more available provider slots comprising MD or Nurse Practitioner (NP) time slots, are received from the EHR system 102 based on the received request upon obtaining the treatment information, the provider information, the resource information, or any combination thereof. For example, the one or more available slots may be from 4:30 PM to 5:00 PM, 6:00 PM to 6:30 PM, and the like. In an embodiment of the present disclosure, the treatment information, the provider information, the resource information, and the one or more available slots are obtained in real-time.

At step 808, one or more optimal treatment times for the treatment date and alternate treatment date for the treatment profile of the patient are determined based on the received request, the obtained treatment information, the obtained provider information, the obtained resource information, or any combination thereof, and the obtained one or more available slots. In an embodiment of the present disclosure, the method 800 includes determining one or more treatment dates, alternate treatment dates and the one or more optimal treatment times for each of the one or more treatment dates and alternate treatment dates for the treatment profile of the patient based on the received request, the obtained treatment information, the obtained provider information, the obtained resource information, or any combination thereof, and the obtained one or more available slots.

In an embodiment of the present disclosure, the method 800 includes determining one or more exact matches or one or more approximate matches corresponding to the one or more optimal treatment times based on the received request, the obtained treatment information, the obtained provider information, the obtained resource information, or any combination thereof, and the obtained one or more available slots. In an embodiment of the present disclosure, the one or more exact matches are the one or more appointment times which exactly correspond to the treatment profile under consideration. Further, the one or more approximate matches are the one or more appointment times which correspond in an approximate manner to the treatment profile under consideration. In an embodiment of the present disclosure, the one or more appointment times may be displayed in a specific way to differentiate the one or more exact matches from the one or more approximate matches.

At step 810, the determined one or more optimal treatment times for the treatment date and alternate treatment date along with the relevant patient information are outputted on user interface screen of the one or more electronic devices 108 associated with the scheduler. In an exemplary embodiment of the present disclosure, the relevant patient information includes patient name, patient identifier, location of treatment, date of the treatment, and the like. In an exemplary embodiment of the present disclosure, the one or more electronic devices 108 may include a laptop computer, desktop computer, tablet computer, smartphone, wearable device, a digital camera and the like. In an embodiment of the present disclosure, the scheduler may use the determined one or more optimal treatment times to schedule the patient's treatment.

In an embodiment of the present disclosure, the method 800 includes receiving historical patient data associated with a patient from the EHR system 102. In an exemplary embodiment of the present disclosure, the historical patient data include service date, a breakdown of different services needed required each treatment, staff schedules, operating hours of the provider, and the like. Further, the method 800 includes generating the set of static optimized Day of the Week (DOW) templates for each DOW based on the received historic patient data by performing a statistical and combinatorial optimization analysis on the received historical patient data. In an embodiment of the present disclosure, the set of static optimized DOW templates include forecasted patient profiles assigned to optimized time slots. The method 800 includes classifying the generated set of static optimized DOW templates into various service types. The method 800 includes categorizing the forecasted patient profiles into individual time stamped services upon classifying the generated set of static optimized DOW templates. In an embodiment of the present disclosure, the forecasted patient profiles are disassembled. Furthermore, the method 800 includes generating a set of optimized and prioritized profiles based on time stamped individual service type, dynamic EHR data, and a profile of the patient to be currently scheduled upon categorizing the forecasted patient profiles. In an embodiment of the present disclosure, the EHR data includes specific details about already scheduled assignments. The dynamically assembled matched profiles combines with the EHR data to generate the set of optimized and prioritized profiles. The method 800 includes determining a set of rank ordered time slots based on the dynamic EHR data, the static optimized DOW template, one or more patient's preferences and one or more different resources required by a specific patient profile by using a patient scheduling-based Artificial Intelligence (AI) model. In an exemplary embodiment of the present disclosure, the one or more patient's preferences include a preferred date, a preferred time, a preferred medical professional, and the like. Further, the method 800 includes outputting the determined set of rank ordered time slots and the generated set of optimized and prioritized profiles on user interface screen of the one or more electronic devices 108 associated with the scheduler.

In determining the set of rank ordered time slots based on the dynamic EHR data, the static optimized DOW template and the one or more different resources required by the specific patient profile by using the patient scheduling-based AI model, the method 800 includes correlating information pertaining to the time slots assigned to various service types derived from the static DOW template with specifics of the dynamic EHR data for a selected clinic for a selected treatment date and alternate treatment date by using the patient scheduling-based AI model. Further, the method 800 includes determining the set of rank ordered time slots based on result of correlation.

In an embodiment of the present disclosure, the method 800 includes obtaining one or more inputs from the EHR system 102. In an exemplary embodiment of the present disclosure, the one or more inputs include patient MRN, treatment schedule date, treatment schedule location, one or more resources, availability of the one or more resources, resource duration, provider schedule, number of patients assigned to each time slot, one or more medical services required by each patient, or any combination thereof. Further, the method 800 includes obtaining an input from the statically optimized DOW template. Furthermore, the method 800 includes determining optimized schedules for specific patient profile under consideration based on the obtained one or more inputs from the EHR system 102 and the obtained input from the statically optimized DOW template.

The AI-based method 800 may be implemented in any suitable hardware, software, firmware, or combination thereof.

Thus, various embodiments of the present system provide a solution to facilitate patient scheduling at the healthcare facility. The computing system 104 obtains and displays patient treatment times to schedulers at treatment facilities. In an embodiment of the present disclosure, the computing system 104 includes mechanisms to obtain and transmit several pieces of information pertinent to provider, treatment, and staff to a server that computes optimized treatment times; receive the optimized times and display them in a user-friendly manner to the scheduler. Further, the computing system 104 dynamically assembles profiles to match a selected treatment profile. The computing system 104 schedules patient appointments at cancer treatment facilities using EHR or EMR software. This is static scheduling where just the availability of resources for a given day is provided to the scheduler and the scheduler simply selects an appointment time based on the patient's preference and/or the scheduler's perception of best option from available times. The computing system 104 considers several other factors as outlined above, to dynamically put together required service types and selects most optimal time based on that. The present disclosure includes a novel way of identifying optimal future patient appointment times for cancer treatment, taking into consideration details such as, but not limited to, historical treatment profile data containing the service date and a breakdown of the different services needed by each treatment, staff schedules, operating hours of the provider and the like is presented. The identified optimal appointment times may be presented to an end user using a graphical user interface as part of a computer program. In an embodiment of the present disclosure, the computing system 104 stores information derived from a static optimization of forecasted treatment profiles for a specific day of the week, known by those familiar with the art as a DOW template, in the form of time stamped service types, and at the time of scheduling, allows the stored information to be retrieved in real time to dynamically assemble one or more profiles matching the selected patient profile. This may further be combined with actual schedule information obtained from EHR system 102 for a specific future date, in an intelligent fashion to produce prioritized and optimized schedules for specific treatment profiles.

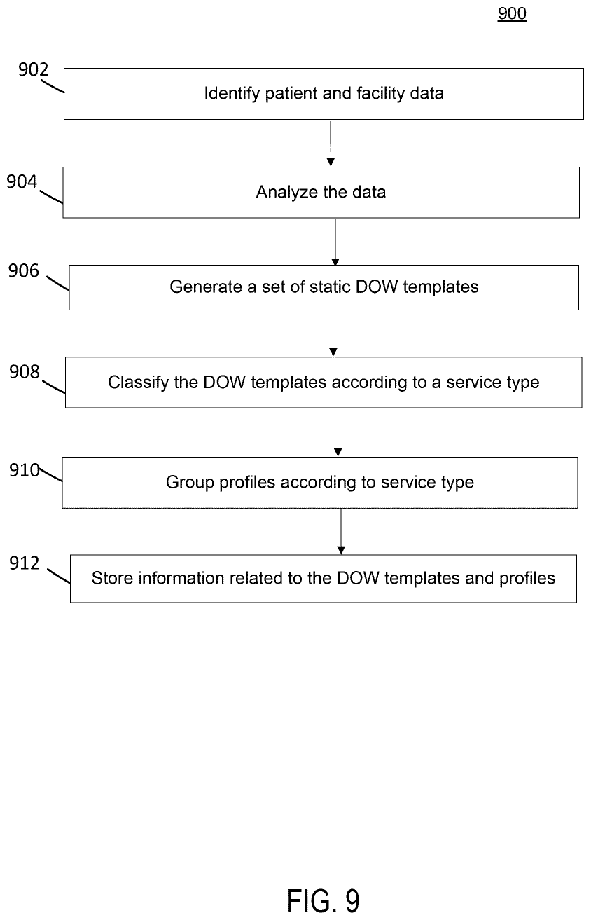

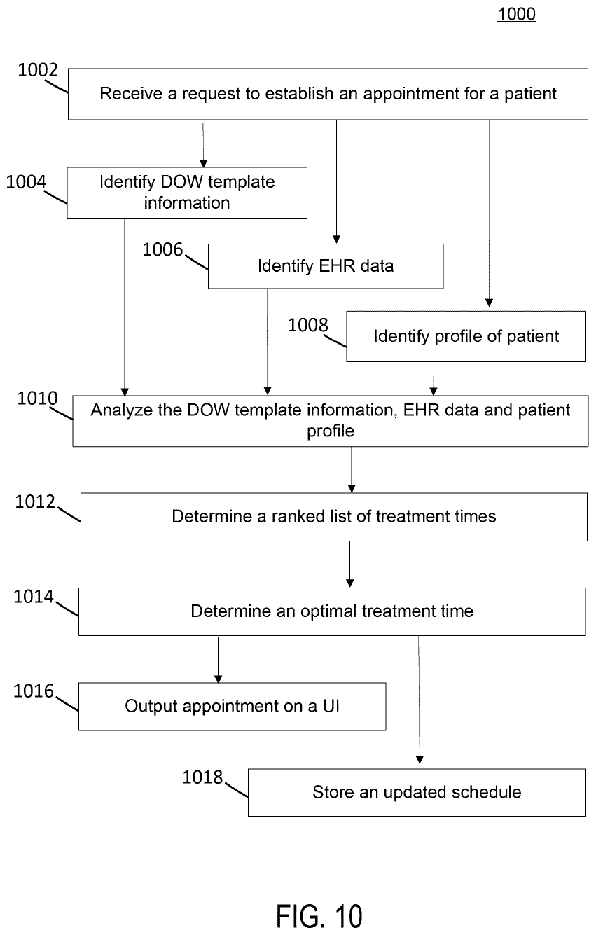

Turning to FIGS. 9 and 10, provided are non-limiting example embodiments for generating a DOW template and utilizing such DOW template, along with dynamic EHR data, inter alia, to provide a schedule output on a UI, as discussed above at least in paragraphs 0030-0071, and depicted in at least FIGS. 2-8. Accordingly, Process 900 of FIG. 9 provides the computerized operations for generating a DOW template(s) and storing patient-related and/or schedule-related information for use by a processor(s) and/or device in Process 1000 of FIG. 10 for automatically, in real-time, scheduling a patient and outputting a result within a UI for use by a user such that allocatable resources can be allotted to patients and facilities in a manner to enable optimal health-care treatment of the patient and optimal use of practitioner and facility resources as discussed herein.

In FIG. 9, in some embodiments, Process 900 begins with Step 902 where patient and facility data is identified. Such identification operation is discussed above at least in paragraphs 0030-0033, 0048, 0052, 0054, 0055, 0067 and 0071, and in FIGS. 4, 5, 6 and 8, inter alia.

According to some embodiments, patient data can include, but is not limited to, service date, a breakdown of different services needed for each treatment, staff schedules, operating hours of the provider, and the like, as discussed supra. In some embodiments, patient data can further and/or alternatively include, but not be limited to, patient name, patient ID, treatment profile, treatment date, location of treatment, and the like, as discussed supra.

According to some embodiments, facility data can include, but not be limited to, service schedules, physician information, MD information, service types, equipment information, provider information, provider name, provider location, provider skillset, provider schedule, provider availability, and the like, as discussed supra.

In Step 904, the identified data from Step 904 can be analyzed in order to generate a DOW template(s), as discussed above and below in Step 906. Such analysis is discussed above at least in paragraphs 0054, 0055 and 0067, and FIGS. 4 and 8, inter alia. According to some embodiments, such analysis can involve, but is not limited to, performing a statistical and combinatorial optimization analysis on the identified data, as discussed supra. In some embodiments, as discussed above, such analysis can involve utilizing an AI model.

In Step 906, a set of static DOW templates can be generated. As discussed above, in some embodiments, DOW templates can include, but are not limited to, forecasted patient profiles assigned to optimized time slots for the days of the week. Such generation is discussed above at least in paragraphs 0044, 0048, 0049, 0050, 0054-0060 and 0067-0071, and FIGS. 4-6 and 8, inter alia.

In Step 908, the DOW templates can be classified according to a service type. Such classification operation includes classifying constituent service types into time-stamped service types and places the time-stamped service types in appropriate service buckets, as is discussed above at least in paragraphs 0048, 0054, 0059, 0067 and 0068, and FIGS. 4, 6 and 8, inter alia.

In Step 910, profiles associated with the DOW templates can be grouped, for example, by disassembling the information comprised within each DOW template and grouping them into time-stamped individual service types. Such grouping is discussed above at least in paragraphs 0048, 0054, 0055, 0059, 0067 and 0068, and FIGS. 4 and 5, inter alia.

And, in Step 912, the information from the proceeding steps, inclusive of the information related to the DOW templates and profiles, can be stored for such purposes of allocating resources to patients and facilities in a manner to enable optimal health-care treatment of the patient and optimal use of practitioner and facility resources as discussed herein. Such storage, and utilization thereof, are discussed herein at least in paragraphs 0037, 0048, 0051, 0054, 0055, 0067-0071 and 0081-0091, and FIGS. 1, 2, 4-8 and 10, inter alia.

Turning to FIG. 10, similar to FIG. 8 discussed supra, depicted and discussed herein are steps for performing patient scheduling at the healthcare facility and outputting a scheduling result (as depicted in at least FIGS. 3A-3B and 7, discussed supra).

In some embodiments, Process 1000 begins with Step 1002, where a request to establish an appointment for a patient is received, as is discussed above at least in paragraphs 0007, 0008, 0031, 0033, 0037, 0038, 0048, 0061, 0065 and 0067-0071, and FIGS. 4, 6 and 8, inter alia. For example, a request is received from one or more electronic devices 108 associated with a scheduler to schedule an appointment of a patient for a treatment profile.

In Step 1004, DOW template information can be identified, which can include, but is not limited to, information indicating a current utilization (e.g., schedule) of resources at the facility (e.g., as per equipment, provider and/or doctor/MD, for example), as discussed supra. Support for such identification is as discussed above at least in paragraphs 0007, 0008, 0039, 0040, 0048, 0049, 0050, 0051, 0054-0059, 0062, 0063 and 0067-0071, and FIGS. 3A-8, inter alia.

In Step 1006, EHR data can be identified as discussed above at least in paragraphs 0007, 0008, 0039, 0040, 0048, 0049, 0050, 0051, 0054-0059, 0062, 0063 and 0067-0071, and FIGS. 3A-8, inter alia. Such EHR data can correspond to, but is not limited to, details about already scheduled assignments at the facility (e.g., available time slots for MDs, NPs, and the like, which can be received from the EHR system 102.

In Step 1008, a profile of patient, and corresponding information/data can be identified. In some embodiments, as discussed above, such identified information/data/metadata can include, but is not limited to, a patient ID of the patient, a treatment profile of the patient, a treatment date, an alternate treatment date, and the like. Support for such identification is as discussed above at least in paragraphs 0007, 0008, 0039, 0040, 0048, 0049, 0050, 0051, 0054-0059, 0062, 0063 and 0067-0071, and FIGS. 3A-8, inter alia

In some embodiments, as discussed above at least in paragraphs 0061-0063, inter alia, such information from Steps 1004-1008 can be at least in part provided within and/or as part of the request in Step 1002.

In Step 1010, the information related to the DOW template(s), EHR data and patient profiles identified in the above steps can be analyzed to identify data for use in further processes, such as for ranking schedule and resource options, as discussed above at least in paragraphs 0007, 0008, 0055-0060, 0064, 0065 and 0067-0071, and FIGS. 6-8, inter alia, and in the subsequent steps of Process 1000.

In Step 1012, a ranked list of treatment times and profiles are determined using such factors as DOW templates, EHR data and patient profiles, as discussed above at least in paragraphs 0007, 0008, 0037, 0049, 0050, 0059, 0067 and 0068, and FIGS. 6-8, inter alia.

In Step 1014, an optimal treatment time is determined based on such factors as the received request, treatment information, provider information, resource information, and the like, or some combination thereof, as discussed above at least in paragraphs 0007, 0008, 0041-0044, 0055-0060, 0064, 0065 and 0067-0071, and FIGS. 6-8, inter alia.

In Step 1016, an appointment and corresponding information are output for display to a scheduler within a user interface (UI), as discussed above at least in paragraphs 0007, 0008, 0045-0048, 0060 and 0066-0071, and FIGS. 3A-3B, 7 and 8, inter alia.

And, in Step 1018, an updated schedule is stored (e.g., based on the appointment from Step 1016 and any scheduler input, if applicable), as discussed above at least in paragraphs 0007, 0008, 0037, 0048, 0051, 0052, 0054-0059 and 0067-0071, and FIGS. 1, 2 and 4-8, inter alia.

The written description describes the subject matter herein to enable any person skilled in the art to make and use the embodiments. The scope of the subject matter embodiments is defined by the claims and may include other modifications that occur to those skilled in the art. Such other modifications are intended to be within the scope of the claims if they have similar elements that do not differ from the literal language of the claims or if they include equivalent elements with insubstantial differences from the literal language of the claims.

The embodiments herein can comprise hardware and software elements. The embodiments that are implemented in software include but are not limited to, firmware, resident software, microcode, etc. The functions performed by various modules described herein may be implemented in other modules or combinations of other modules. For the purposes of this description, a computer-usable or computer readable medium can be any apparatus that can comprise, store, communicate, propagate, or transport the program for use by or in connection with the instruction execution system, apparatus, or device.

The medium can be an electronic, magnetic, optical, electromagnetic, infrared, or semiconductor system (or apparatus or device) or a propagation medium. Examples of a computer-readable medium include a semiconductor or solid-state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-R/W) and DVD.

Input/output (I/O) devices (including but not limited to keyboards, displays, pointing devices, etc.) can be coupled to the system either directly or through intervening I/O. Network adapters may also be coupled to the system to enable the data processing system to become coupled to other data processing systems or remote printers or storage devices through intervening private or public networks. Modems, cable modem and Ethernet cards are just a few of the currently available types of network adapters.

A representative hardware environment for practicing the embodiments may include a hardware configuration of an information handling/computer system in accordance with the embodiments herein. The system herein comprises at least one processor or central processing unit (CPU). The CPUs are interconnected via system bus 308 to various devices such as a random-access memory (RAM), read-only memory (ROM), and an input/output (I/O) adapter. The I/O adapter can connect to peripheral devices, such as disk units and tape drives, or other program storage devices that are readable by the system. The system can read the inventive instructions on the program storage devices and follow these instructions to execute the methodology of the embodiments herein.

The system further includes a user interface adapter that connects a keyboard, mouse, speaker, microphone, and/or other user interface devices such as a touch screen device (not shown) to the bus to gather user input. Additionally, a communication adapter connects the bus to a data processing network, and a display adapter connects the bus to a display device which may be embodied as an output device such as a monitor, printer, or transmitter, for example.

A description of an embodiment with several components in communication with each other does not imply that all such components are required. On the contrary, a variety of optional components are described to illustrate the wide variety of possible embodiments of the disclosure. When a single device or article is described herein, it will be apparent that more than one device/article (whether or not they cooperate) may be used in place of a single device/article. Similarly, where more than one device or article is described herein (whether or not they cooperate), it will be apparent that a single device/article may be used in place of the more than one device or article, or a different number of devices/articles may be used instead of the shown number of devices or programs. The functionality and/or the features of a device may be alternatively embodied by one or more other devices which are not explicitly described as having such functionality/features. Thus, other embodiments of the disclosure need not include the device itself.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments. Also, the words “comprising,” “having,” “containing,” and “including,” and other similar forms are intended to be equivalent in meaning and be open-ended in that an item or items following any one of these words is not meant to be an exhaustive listing of such item or items or meant to be limited to only the listed item or items. It must also be noted that as used herein and in the appended claims, the singular forms “a,” “an,” and “the” include plural references unless the context clearly dictates otherwise.

Finally, the language used in the specification has been principally selected for readability and instructional purposes, and it may not have been selected to delineate or circumscribe the inventive subject matter. It is therefore intended that the scope of the disclosure be limited not by this detailed description, but rather by any claims that issue on an application based here on. Accordingly, the embodiments of the present disclosure are intended to be illustrative, but not limiting, of the scope of the disclosure, which is set forth in the following claims.

1-20. (canceled)

21. A method comprising:
receiving, by an application running on a computing device, a scheduling request related to a patient and a facility, the request corresponding to a treatment needed by the patient at the facility;
analyzing, by the application, stored data representing information related to a day of week (DOW) template, electronic health records (EHR) data and a profile of the patient, the DOW template being derived from historic patient data for the facility by performing a statistical and combinatorial optimization analysis on the historical patient data and comprising a calculated allocation of resources at the facility according to a time and date, the profile comprising current information for the patient related to the needed treatment;
determining, by the application, based on the analysis, in real time, a ranked list of optimal treatment times and dates for the patient at the facility, the optimal treatment times and dates corresponding to an availability of resources at the facility that enable performance of the treatment while ensuring other resources remain available for other patients; and
providing, by the application, to a user of the application, an output for visible display on the computing device within a user interface (UI), the output comprising electronic information related to the patient from the profile and the ranked list of optimal treatment times and dates for selection by the user via the application.

22. The method of claim 21, further comprising:
generating a treatment profile for the patient based on the EHR data and the profile; and
performing the optimal treatment times and dates determination based on the treatment profile, such that at least one of a treatment type, duration, order or service indicated within the treatment profile dictates at least one of a time or date of the determined treatment.

23. The method of claim 21, further comprising:
receiving the selection, by the application, from the user of a treatment time and date from the ranked list of optimal treatment times and dates; and
storing an optimized schedule in a database based on the selection.

24. The method of claim 23, further comprising the selection impacting resource allocation and availability at the facility for the selected treatment time and date and other treatment times and dates.

25. The method of claim 21, further comprising:
obtaining historical information related to a set of patients and the facility;
analyzing the historical information;
generating a set of DOW templates for the facility; and
classifying each DOW template according to a service type.

26. The method of claim 25, further comprising:
identifying a set of patient profiles for the set of patients from the DOW templates; and
grouping the set of patients profiles according to the service type.

27. The method of claim 26, further comprising the grouping being based further on a time stamp of the service type.

28. The method of claim 26, further comprising:
storing information related to the DOW templates and grouped set of patient profiles; and
performing the analysis based on the stored information.

29. The method of claim 21, further comprising performing the analysis via execution of an artificial intelligence (AI) model.

30. A device comprising;
a processor configured to:
receive, by an application running on the device, a scheduling request related to a patient and a facility, the request corresponding to a treatment needed by the patient at the facility;
analyze, by the application, stored data representing information related to a day of week (DOW) template, electronic health records (EHR) data and a profile of the patient, the DOW template being derived from historic patient data for the facility by performing a statistical and combinatorial optimization analysis on the historical patient data and comprising a calculated allocation of resources at the facility according to a time and date, the profile comprising current information for the patient related to the needed treatment;
determine, by the application, based on the analysis, in real time, a ranked list of optimal treatment times and dates for the patient at the facility, the optimal treatment times and dates corresponding to an availability of resources at the facility that enable performance of the treatment while ensuring other resources remain available for other patients; and
provide, by the application, to a user of the application, an output for visible display on the computing device within a user interface (UI), the output comprising electronic information related to the patient from the profile and the ranked list of optimal treatment times and dates for selection by the user via the application.

31. The device of claim 30, wherein the processor is further configured to:
generate a treatment profile for the patient based on the EHR data and the profile; and
perform the optimal treatment times and dates determination based on the treatment profile, such that at least one of a treatment type, duration, order or service indicated within the treatment profile dictates at least one of a time or date of the determined treatment.

32. The device of claim 30, wherein the processor is further configured to:
receive the selection, by the application, from the user of a treatment time and date from the ranked list of optimal treatment times and dates; and
store an optimized schedule in a database based on the selection.

33. The device of claim 32, wherein the processor is further configured such that the selection impacts resource allocation and availability at the facility for the selected treatment time and date and other treatment times and dates.

34. The device of claim 30, wherein the processor is further configured to:
obtain historical information related to a set of patients and the facility;
analyze the historical information;
generate a set of DOW templates for the facility; and
classify each DOW template according to a service type.

35. The device of claim 34, wherein the processor is further configured to:
identify a set of patient profiles for the set of patients from the DOW templates; and
group the set of patients profiles according to the service type.

36. The device of claim 35, wherein the processor is further configured such that the grouping is based further on a time stamp of the service type.

37. The device of claim 35, wherein the processor is further configured to:
store information related to the DOW templates and grouped set of patient profiles; and
perform the analysis based on the stored information.

38. A non-transitory computer-readable storage medium tangibly encoded with computer-executable instructions, that when executed by a processor, perform a method comprising:
receiving, by an application running on a computing device, a scheduling request related to a patient and a facility, the request corresponding to a treatment needed by the patient at the facility;
analyzing, by the application, stored data representing information related to a day of week (DOW) template, electronic health records (EHR) data and a profile of the patient, the DOW template being derived from historic patient data for the facility by performing a statistical and combinatorial optimization analysis on the historical patient data and comprising a calculated allocation of resources at the facility according to a time and date, the profile comprising current information for the patient related to the needed treatment;
determining, by the application, based on the analysis, in real time, a ranked list of optimal treatment times and dates for the patient at the facility, the optimal treatment times and dates corresponding to an availability of resources at the facility that enable performance of the treatment while ensuring other resources remain available for other patients; and
providing, by the application, to a user of the application, an output for visible display on the computing device within a user interface (UI), the output comprising electronic information related to the patient from the profile and the ranked list of optimal treatment times and dates for selection by the user via the application.

39. The non-transitory computer-readable storage medium of claim 38, further comprising:
receiving the selection, by the application, from the user of a treatment time and date from the ranked list of optimal treatment times and dates; and
storing an update to the DOW template based on the selection, such that an optimized schedule is stored in a database.

40. The non-transitory computer-readable storage medium of claim 39, further comprising the selection impacting resource allocation and availability at the facility for the selected treatment time and date and other treatment times and dates.
